(19) United States
(12) Patent Application Publication
(10) Pub. No.: US 2026/0074016 A1
(43) Pub. Date: Mar. 12, 2026

(54) INFORMATION PROCESSING APPARATUS, INFORMATION PROCESSING METHOD, AND COMPUTER PROGRAM PRODUCT

(71) Applicant: KABUSHIKI KAISHA TOSHIBA, Tokyo (JP)

(72) Inventors: Sunao YOTSUTSUJI, Kawasaki (JP); Masaaki TAKADA, Kashiwa (JP); Hisashi YAMADA, Kawasaki (JP); Yoshitake SANO, Adachi (JP); Tomomi ANDO, Ota (JP); Mitsuko ISHIHARA, Setagaya (JP)

(73) Assignee: KABUSHIKI KAISHA TOSHIBA, Tokyo (JP)

(21) Appl. No.: 19/267,751

(22) Filed: Jul. 14, 2025

(30) Foreign Application Priority Data
Sep. 11, 2024 (JP) 2024-157120

(51) Int. Cl. G16B 25/10; G16B 40/20

(52) U.S. Cl. CPC G16B 25/10; G16B 40/20

(57) ABSTRACT

An information processing apparatus according to an embodiment includes a hardware processor connected to a memory. The hardware processor estimates a first disease incidence probability of a disease based on first information about expression levels of one or more types of biomarkers of a specimen. The hardware processor estimates a second disease incidence probability of a disease type based on the first information and the first disease incidence probability.

### CROSS-REFERENCE TO RELATED APPLICATIONS

This application is based upon and claims the benefit of priority from Japanese Patent Application No. 2024-157120, filed on Sep. 11, 2024; the entire contents of which are incorporated herein by reference.

### FIELD

Embodiments described herein relate generally to an information processing apparatus, an information processing method, and a computer program product.

### BACKGROUND

The technique for estimating a disease, such as cancer, with focusing on the expression level of miRNAs (microRNAs) in a specimen such as blood has been known. For example, a technique has been proposed that estimates the possibility of cancer incidence by comparing the expression level of miRNAs obtained from a subject with the expression level of miRNAs obtained from a healthy person.

In addition, a technique has been known that generates information about the presence or absence of incidence of plural diseases by inputting information about plural types of biomarkers to one learned model.

However, the classification problem of directly estimating a specific disease from a specimen of a subject who may be suffering from any of cancers is difficult to solve, and the estimation performance may degrade. Additionally, in the case of estimating the incidence probability of a specific type of cancer, if the subject has a low incidence probability of the specific type of cancer, the estimation performance as to whether or not the subject is suffering from cancer itself may degrade. Thus, in the prior art, the estimation performance of the disease may degrade.

### BRIEF DESCRIPTION OF THE DRAWINGS

FIG. 1 is a schematic diagram of an information processing apparatus according to an embodiment;

FIG. 2A is an explanatory diagram of biomarker information according to the embodiment;

FIG. 2B is an explanatory diagram of specimen related information according to the embodiment;

FIG. 2C is an explanatory diagram of a feature according to the embodiment;

FIG. 3 is an explanatory diagram of estimation of a first disease incidence probability according to the embodiment;

FIG. 4 is an explanatory diagram of the first disease incidence probability according to the embodiment;

FIG. 5 is an explanatory diagram of estimation of a second disease incidence probability according to the embodiment;

FIG. 6 is an explanatory diagram of the second disease incidence probability according to the embodiment;

FIG. 7 is a flowchart illustrating a procedure of information processing according to the embodiment; and

FIG. 8 is a hardware configuration diagram according to the embodiment.

### DETAILED DESCRIPTION

An information processing apparatus according to one embodiment includes a hardware processor connected to a memory. The hardware processor is configured to estimate a first disease incidence probability of a disease based on first information about expression levels of one or more types of biomarkers of a specimen. The hardware processor is configured to estimate a second disease incidence probability of a disease type based on the first information and the first disease incidence probability.

Hereinafter, an information processing method, an information processing apparatus, and a computer program product according to an embodiment will be described in detail with reference to the accompanying drawings.

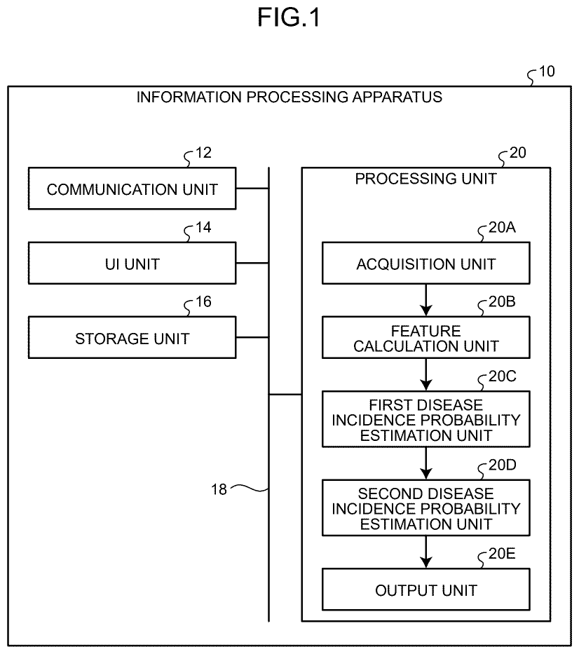

FIG. 1 is a schematic diagram of an example of an information processing apparatus 10 according to the present embodiment.

The information processing apparatus 10 executes processing of estimating the incidence probability of a disease or each of disease types of a subject based on biomarker information of a specimen of the subject. The information processing apparatus 10 includes one or more dedicated or general-purpose computers.

The information processing apparatus 10 includes a communication unit 12, a user interface (UI) unit 14, a storage unit 16, and a processing unit 20. The communication unit 12, the UI unit 14, the storage unit 16, and the processing unit 20 are communicably connected via a bus 18 or the like.

The communication unit 12 communicates with an external information processing apparatus or the like via a network or the like.

The UI unit 14 has a display function of displaying various types of information and an input function of receiving an operation input by a user. The display function is, for example, a display, a projection device, or the like. The input function is, for example, a pointing device such as a mouse and a touch pad, a keyboard, or the like. A touch panel that integrates the display function and the input function may be used.

It is sufficient for the UI unit 14 to be communicably connected to the processing unit 20 in a wired or wireless manner. The UI unit 14 may be provided outside the information processing apparatus 10, and the UI unit 14 and the processing unit 20 may be connected via a network or the like.

The storage unit 16 stores various types of data. The storage unit 16 may be provided outside the information processing apparatus 10. In addition, the storage unit 16 and/or one or more functional units included in the processing unit 20 to be described later may be installed in the external information processing apparatus communicably connected to the information processing apparatus 10 via a network or the like.

The processing unit 20 executes information processing in the information processing apparatus 10. The processing unit 20 includes an acquisition unit 20A, a feature calculation unit 20B, a first disease incidence probability estimation unit 20C, a second disease incidence probability estimation unit 20D, and an output unit 20E.

The acquisition unit 20A, the feature calculation unit 20B, the first disease incidence probability estimation unit 20C, the second disease incidence probability estimation unit 20D, and the output unit 20E are implemented by, for example, one or more processors. For example, each of the above-described units may be implemented by causing a processor such as a central processing unit (CPU) to execute a program, namely, by software. Each of the above-described units may be implemented by a processor such as a dedicated IC or circuit, namely, hardware. Each of the above-described units may be implemented by a combination of software and hardware. In a case where a plurality of processors are used, each processor may implement one of the units, or may implement two or more of the units.

The acquisition unit 20A acquires one or more types of biomarker information of the specimen and specimen related information.

The specimen refers to a sample derived from a living body of the subject. The specimen may be referred to as a biological sample or the like. Examples of the specimen include, but are not limited to, blood, serum, plasma, urine, saliva, gastric fluid, and the like.

The subject refers to a living body subject to the estimation of the incidence probability of the disease by the information processing apparatus 10. The subject is, for example, a person, but may be a living organism other than a person. In the present embodiment, it is assumed that the subject is a person.

The disease refers to a pathological condition of a subject. The disease subject to the estimation by the information processing apparatus 10 of the present embodiment may be either a benign disease or a malignant disease. The benign disease include benign tumors and various benign diseases of each organ and each site of the living body. Examples of the benign disease include, but are not limited to, a benign breast disease, benign prostate disease, benign pancreatic disease, and the like. The malignant diseases include malignant tumors, i.e., cancers. The cancer is classified into a plurality of types according to its classification criteria. When the cancer is classified by site, examples of a cancer type include, but are not limited to, breast cancer, prostate cancer, pancreatic cancer, biliary tract cancer, large bowel cancer, gastric cancer, esophageal cancer, ovarian cancer, lung cancer, pancreatic cancer, bile duct cancer, uterine cancer, cervical cancer, liver cancer, leukemia, bladder cancer, and malignant brain tumor. In addition, the cancer type may include a type defined from further detailed classification of the above-described classification by site. For example, the cancer type may include invasive pancreatic ductal carcinoma, acinar cell carcinoma, and the like, which are further detailed classifications of pancreatic cancer, in addition to the pancreatic cancer, which is cancer of the site “pancreas”.

The present embodiment will be described on the assumption that cancer is the disease subject to the estimation of the disease incidence probability by the information processing apparatus 10, and that a cancer type is the disease type subject to the estimation of the disease incidence probability.

The biomarker information is information indicating the expression level of a biomarker (biological index) of a specimen. The expression level is represented by, for example, concentration or the like. Examples of the biomarker include, but are not limited to, miRNAs (micro-RNAs), plasma LDL (low-density lipoprotein), p53 genes, matrix metalloprotease, KRAS genes, and the like.

In the present embodiment, a case where the biomarker is miRNAs will be described.

miRNAs are functional nucleic acids composed of a single-stranded RNA having a base length of 21 to 25. The miRNAs have a function of suppressing translation of various genes having a target site complementary to themselves, and the miRNAs are known to control basic biological functions such as cell development, differentiation, proliferation, and cell death. More than 2500 types of human miRNAs have been found.

As described above, in the present embodiment, it is assumed that the disease subject to the estimation of the disease incidence probability by the information processing apparatus 10 is cancer, and that the disease type subject to the estimation of the disease incidence probability is the cancer type. In addition, in the present embodiment, the case where the biomarker is miRNAs (micro-RNAs) will be described. However, each of the disease and the disease type subject to the estimation of the disease incidence probability by the information processing apparatus 10 of the present embodiment, and the biomarker used for the estimation thereof is not limited thereto.

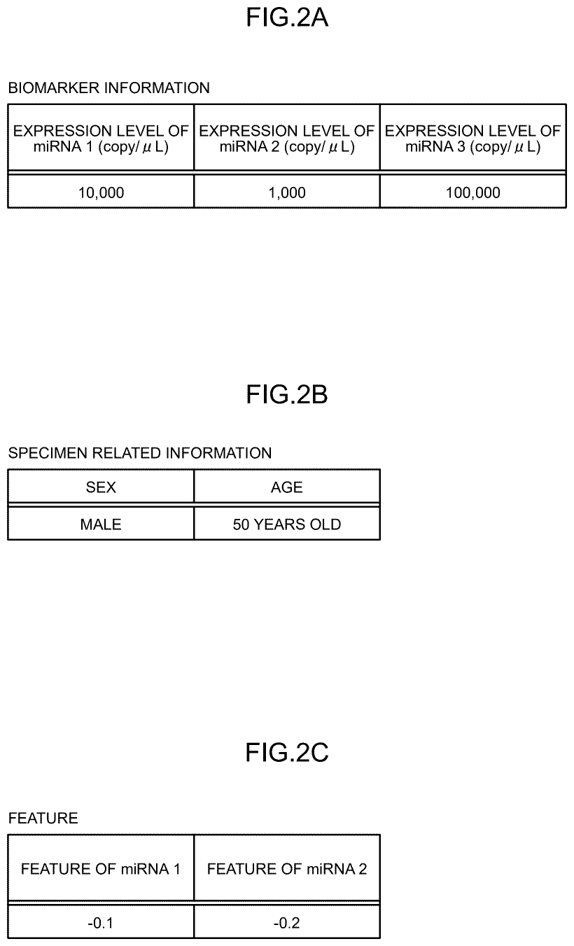

FIG. 2A is an explanatory diagram of an example of the biomarker information acquired by the acquisition unit 20A. In FIG. 2A, the expression level of each of three types of miRNAs 1 to 3 is illustrated as an example. The numerical values 1 to 3 following the miRNAs are identification information of the miRNAs. It is sufficient for these miRNAs 1 to 3 to be different types of miRNAs.

For example, the acquisition unit 20A acquires the measured value of the expression level of each of the types of miRNAs as types of biomarker information in the specimen. The expression level is represented by, for example, concentration. FIG. 2A illustrates, as an example, a case where the acquisition unit 20A acquires the concentration of each of the three types of miRNAs in the specimen as the biomarker information.

The number of types of miRNAs acquired by the acquisition unit 20A is not limited to three and is only required to be one or more.

The method for measuring the miRNAs is not limited. For example, the acquisition unit 20A acquires, as the biomarker information, an miRNA expression level measured by at least one of a polymerase chain reaction (PCR) method, a loop-mediated isothermal amplification (LAMP) method, a microarray method, a nanostring method, and a next-generation sequencing method.

The specimen related information refers to information about the specimen. For example, the specimen related information includes at least one of information on another type of biomarker other than the biomarker acquired as the biomarker information in the specimen, and information about the subject from which the specimen is collected. The information on the subject includes information indicating at least one of the age, height, weight, medical history, medication history, and various test results of the subject.

FIG. 2B is an explanatory diagram of an example of the specimen related information.

For example, the acquisition unit 20A acquires the specimen related information including the sex “male” and the age “50 years old” of the subject.

Returning to FIG. 1, the description will be continued.

The feature calculation unit 20B calculates the feature of the expression level of the biomarker represented by the biomarker information acquired by the acquisition unit 20A.

The feature refers to a relative index of the expression level of the biomarker with respect to a reference index. The reference index is, for example, a reference variance value, a statistical value, or the like. Specifically, the feature is calculated by correcting the expression level of the biomarker to a relative value with respect to a reference expression level and then obtaining a relative index with respect to the reference index. The feature calculation unit 20B may use one of the types of biomarker information acquired by the acquisition unit 20A as the reference expression level, or may use a value stored in advance.

For example, the feature calculation unit 20B calculates the feature by executing, in an optional order, any one or more of correction based on arithmetic operation using the reference expression level, standardization, whitening, addition of a new feature based on arithmetic operation of a logarithmic value, and correction based on arithmetic operation using the specimen related information, on the expression levels of the plurality of types of biomarkers represented by the types of biomarker information acquired by the acquisition unit 20A. For the standardization, a standardization method using an average value or a variance value as a reference may be employed.

FIG. 2C is an explanatory diagram of an example of the feature.

In FIG. 2C, an example is illustrated in which the feature calculation unit 20B calculates the feature of each of the two types of miRNAs illustrated in FIG. 2C from the expression levels of three types of miRNAs represented by three types of biomarker information illustrated in FIG. 2A.

For example, the feature calculation unit 20B sets the miRNA 1 and the miRNA 2 as miRNAs for classification, and sets the miRNA 3 as the miRNA for the reference expression level. Then, the feature calculation unit 20B obtains a division result obtained by dividing the expression level of each of the miRNA 1 and the miRNA 2 that are the miRNAs for classification by the expression level of the miRNA 3 that is the reference expression level. The division corresponds to correction based on the arithmetic operation using the reference expression level.

The feature calculation unit 20B calculates each common logarithmic value for each of the division results obtained by dividing the expression level of each of the miRNA 1 and the miRNA 2 by the concentration of the miRNA 3. The feature calculation unit 20B then calculates the feature of each of the miRNA 1 and the miRNA 2 by standardizing the calculated common logarithmic value of each of the miRNA 1 and the miRNA 2 using the average value/variance value each serving as the reference.

Specifically, as illustrated in FIG. 2A, assume that the expression level of the miRNA 1 is “10,000 copy/μL” and the concentration of the miRNA 3 is “100,000 copy/μL”. The division result obtained by dividing the expression level of the miRNA 1 by the expression level of the miRNA 3 that is the reference expression level is “0.1”, and the common logarithmic value of the division result “0.1” is “−1”. When the feature calculation unit 20B standardizes the common logarithmic value “−1” using the average value “0” and the variance value “100” as the references, the feature calculation unit 20B calculates “−0.1” as the feature of the expression level of the miRNA 1. By performing a similar calculation for the miRNA 2, the feature calculation unit 20B calculates “−0.2” as the feature of the expression level of the miRNA 2.

Note that the feature calculation unit 20B may calculate the feature of each biomarker information acquired by the acquisition unit 20A by performing plural types of calculation processing on the plural types of biomarker information. For the calculation processing, numerical calculation, statistical processing, or a machine learning method may be used, and information related to plural types of other biomarker information may be used in them to adjust the calculation processing method in advance. In addition, each of the types of biomarker information may be used without a change as the feature of each biomarker information.

Returning to FIG. 1, the description will be continued.

The first disease incidence probability estimation unit 20C estimates the first disease incidence probability of the disease based on first information about the expression levels of one or more types of biomarkers of the specimen.

The first disease incidence probability is information indicating the disease incidence probability of the disease of the subject. The first disease incidence probability indicates the probability that the subject is suffering from the disease. As described above, in the present embodiment, the description is given on the assumption that the disease is cancer. In this case, the first disease incidence probability is information indicating the probability that the subject is suffering from cancer.

The first information is information indicating any one of the one or more types of biomarker expression levels represented by the one or more types of biomarker information acquired by the acquisition unit 20A, and the feature calculated by the feature calculation unit 20B. In the present embodiment, a case where the first disease incidence probability estimation unit 20C uses the feature calculated by the feature calculation unit 20B as the first information, will be described as an example.

In addition, the first disease incidence probability estimation unit 20C may estimate the first disease incidence probability based on the first information and the specimen related information. In the present embodiment, a mode in which the first disease incidence probability estimation unit 20C estimates the first disease incidence probability based on the feature calculated by the feature calculation unit 20B and the specimen related information acquired by the acquisition unit 20A, will be described as an example.

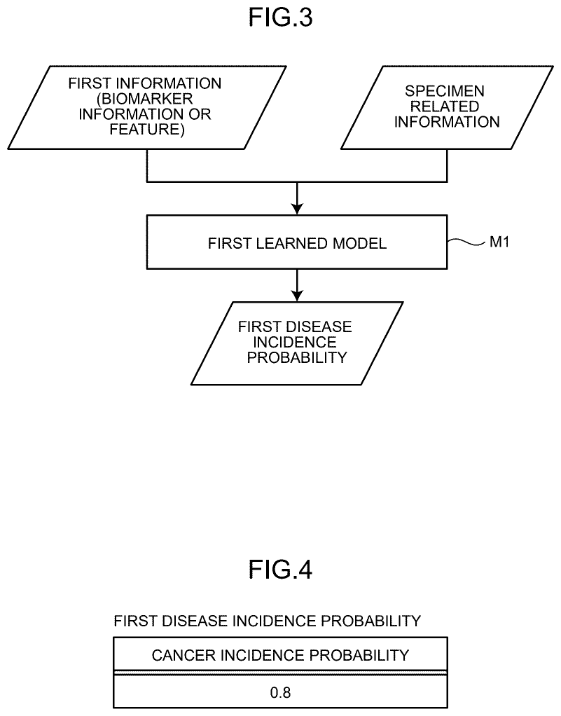

FIG. 3 is an explanatory diagram of an example of the estimation of the first disease incidence probability by the first disease incidence probability estimation unit 20C.

The first disease incidence probability estimation unit 20C obtains the first disease incidence probability as an output from a first learned model M1 by inputting the feature for each of the one or more types of biomarker information calculated by the feature calculation unit 20B and the specimen related information acquired by the acquisition unit 20A, to the first learned model M1.

The first learned model M1 is any of the following machine learning models: a learned logistic regression model, a linear regression model, a generalized linear regression model, a decision tree model, a gradient boosting model, a neural network model, and the like. It is sufficient for the first learned model M1 to be a learned model that has been learned in advance so as to output the first disease incidence probability in response to input of the feature and the specimen related information.

Note that, in a case where the biomarker information is used as the first information, the first learned model M1 may be a learned model that has been learned in advance to output the first disease incidence probability in response to input of the biomarker information and the specimen related information.

In addition, in a case where the first disease incidence probability estimation unit 20C estimates the first disease incidence probability only from the first information, the first learned model M1 may be a learned model that has been learned in advance to output the first disease incidence probability in response to input of the first information.

FIG. 4 is an explanatory diagram of an example of the first disease incidence probability.

For example, assume that the first disease incidence probability estimation unit 20C inputs the specimen related information illustrated in FIG. 2B and the feature illustrated in FIG. 2C to the first learned model M1. In this case, the first disease incidence probability estimation unit 20C estimates the cancer incidence probability illustrated in FIG. 4 as the first disease incidence probability of example, as the output from the first learned model M1.

Returning to FIG. 1, the description will be continued.

The second disease incidence probability estimation unit 20D estimates the second disease incidence probability of the disease type based on the first information and the first disease incidence probability.

The second disease incidence probability is information indicating the disease incidence probability of the disease type of the subject. The second disease incidence probability is information indicating the probability that the subject is suffering from the disease type for each of the one or more types of diseases. As described above, in the present embodiment, the description is given assuming that the disease is cancer. In this case, the second disease incidence probability is information indicating the disease incidence probability of each cancer type such as breast cancer, prostate cancer, pancreatic cancer, biliary tract cancer, large bowel cancer, gastric cancer, esophageal cancer, ovarian cancer, lung cancer, pancreatic cancer, bile duct cancer, uterine cancer, cervical cancer, liver cancer, leukemia, bladder cancer, and malignant brain tumor. The second disease incidence probability estimation unit 20D may estimate the second disease incidence probability of one specific disease type, or may estimate the second disease incidence probability of each of plural disease types.

As described above, the first information may be any one of the one or more types of biomarker expression levels represented by the one or more types of biomarker information acquired by the acquisition unit 20A, and the feature calculated by the feature calculation unit 20B. In the present embodiment, a case where the second disease incidence probability estimation unit 20D uses the feature calculated by the feature calculation unit 20B as the first information, will be described as an example.

In addition, the second disease incidence probability estimation unit 20D may estimate the second disease incidence probability based on the first information, the first disease incidence probability, and the specimen related information. In the present embodiment, a mode will be described as an example, in which the second disease incidence probability estimation unit 20D estimates the second disease incidence probability based on the first disease incidence probability estimated by the first disease incidence probability estimation unit 20C, the feature calculated by the feature calculation unit 20B, and the specimen related information acquired by the acquisition unit 20A.

FIG. 5 is an explanatory diagram of an example of the estimation of the second disease incidence probability by the second disease incidence probability estimation unit 20D.

The second disease incidence probability estimation unit 20D obtains the second disease incidence probability as an output from a second learned model M2 by inputting the first disease incidence probability estimated by the first disease incidence probability estimation unit 20C, the feature for each of the expression levels of the one or more types of biomarkers calculated by the feature calculation unit 20B, and the specimen related information acquired by the acquisition unit 20A, to the second learned model M2.

The second learned model M2 is any of the following machine learning models: a learned logistic regression model, a linear regression model, a generalized linear regression model, a decision tree model, a gradient boosting model, a neural network model, and the like. It is sufficient for the second learned model M2 to be a learned model that has been learned in advance to output the second disease incidence probability in response to input of the first disease incidence probability, the feature, and the specimen related information.

Note that, in the case where the biomarker information is used as the first information, the second learned model M2 may be a learned model that has been learned in advance to output the second disease incidence probability in response to input of the first disease incidence probability, the biomarker information, and the specimen related information.

In addition, in a case where the second disease incidence probability estimation unit 20D estimates the second disease incidence probability from the first disease incidence probability and the first information, the second learned model M2 may be a learned model that has been learned in advance to output the second disease incidence probability in response to input of the first disease incidence probability and the first information.

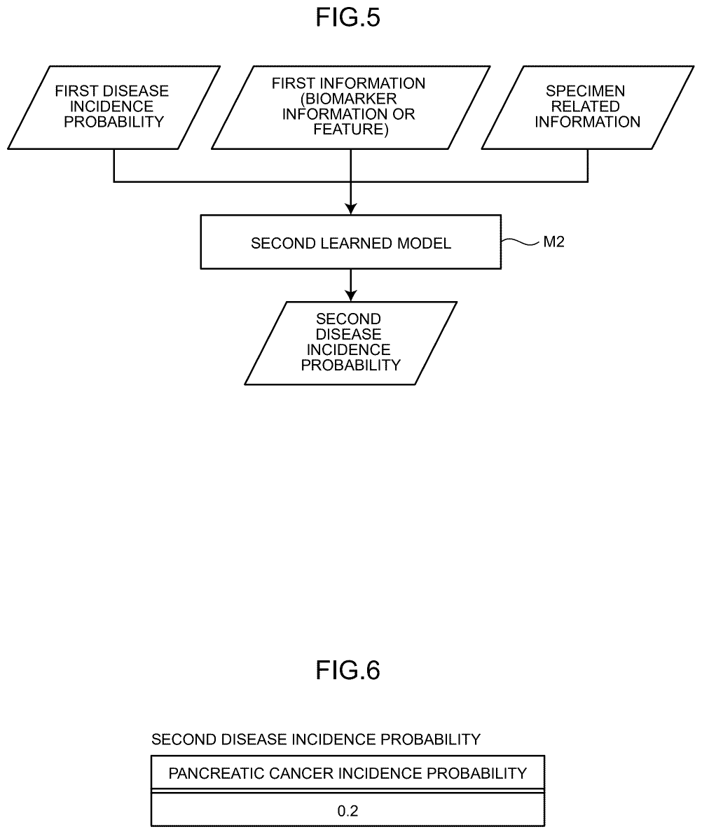

FIG. 6 is an explanatory diagram of an example of the second disease incidence probability.

For example, assume that the second disease incidence probability estimation unit 20D inputs the first disease incidence probability illustrated in FIG. 4, the specimen related information illustrated in FIG. 2B, and the feature illustrated in FIG. 2C to the second learned model M2. In this case, for example, the second disease incidence probability estimation unit 20D estimates the second incidence probability illustrated in FIG. 6 as the output from the second learned model M2. FIG. 6 illustrates pancreatic cancer as the cancer type, and illustrates the incidence probability of pancreatic cancer as the second disease incidence probability. However, as described above, the second disease incidence probability estimated by the second disease incidence probability estimation unit 20D may be an incidence probability of the disease type, and the type is not limited to pancreatic cancer.

Note that FIG. 6 illustrates, as an example, a scene in which the second disease incidence probability estimation unit 20D estimates the second disease incidence probability of one type of cancer. However, as described above, the second disease incidence probability estimation unit 20D may estimate the second disease incidence probability of each of plural types of cancer types.

In this case, the second learned model M2 may be a learned model that outputs the second disease incidence probability of each of the cancer types in response to input of the first probability, the first information, and the specimen related information.

Returning to FIG. 1, the description will be continued.

The output unit 20E outputs, to an output device, output information about the first disease incidence probability estimated by the first disease incidence probability estimation unit 20C and the second disease incidence probability estimated by the second disease incidence probability estimation unit 20D.

The output device is at least one of the UI unit 14, the storage unit 16, and the external information processing apparatus connected to the information processing apparatus 10 via the communication unit 12. For example, in a case where a display that is a display function of the UI unit 14 is used as the output device, the output unit 20E displays the output information on the display of the UI unit 14. In addition, for example, in a case where the storage unit 16 is used as the output device, the output unit 20E outputs the output information by storing the output information in the storage unit 16. Moreover, for example, in a case where the external information processing apparatus is used as the output device, the output unit 20E output the output information by transmitting the output information to the external information processing apparatus.

It is sufficient for the output information to be information about the first disease incidence probability estimated by the first disease incidence probability estimation unit 20C and the second disease incidence probability estimated by the second disease incidence probability estimation unit 20D.

The output unit 20E outputs output information including the first disease incidence probability estimated by the first disease incidence probability estimation unit 20C and the second disease incidence probability estimated by the second disease incidence probability estimation unit 20D.

Specifically, assume that the first disease incidence probability estimation unit 20C estimates the first disease incidence probability illustrated in FIG. 4, and the second disease incidence probability estimation unit 20D estimates the second disease incidence probability illustrated in FIG. 6. In this case, the output unit 20E outputs output information including “cancer incidence probability” and “0.8” as the first disease incidence probability and “pancreatic cancer incidence probability” and “0.2” as the second disease incidence probability to the output device. In this case, the output unit 20E can output information indicating that the incidence probability of pancreatic cancer, which is a specific type of cancer, is low, whereas the incidence probability of any type of cancer other than pancreatic cancer is high.

In addition, the output unit 20E may output, to the output device, output information including at least one of a first determination result on the presence or absence of incidence of the disease based on the first disease incidence probability and a second determination result on the presence or absence of incidence of a disease type based on the second disease incidence probability.

For example, in a case where the first disease incidence probability estimated by the first disease incidence probability estimation unit 20C is equal to or greater than a predetermined first threshold, the output unit 20E generates the first determination result indicating the presence of incidence of cancer in the subject. In addition, in a case where the first disease incidence probability estimated by the first disease incidence probability estimation unit 20C is less than the predetermined first threshold, the output unit 20E generates the first determination result indicating the absence of incidence of cancer in the subject.

The first threshold is, for example, 0.5 or the like, but is not limited to this value. It is sufficient for the first threshold to be determined in advance. In addition, the first threshold may be appropriately changeable according to an operation instruction or the like of the UI unit 14 by the user.

In addition, for example, in a case where the second disease incidence probability estimated by the second disease incidence probability estimation unit 20D is equal to or greater than a predetermined second threshold, the output unit 20E generates the second determination result indicating the presence of incidence of the specific type of cancer in the subject. In addition, in a case where the second disease incidence probability estimated by the second disease incidence probability estimation unit 20D is less than the predetermined second threshold, the output unit 20E generates the second determination result indicating the absence of incidence of the type of cancer in the subject.

The second threshold is, for example, 0.5 or the like, but is not limited to this value. It is sufficient for the second threshold to be determined in advance. In addition, the second threshold may be appropriately changeable according to the operation instruction or the like of the UI unit 14 by the user.

The output unit 20E may output the output information including at least one of the first determination result and the second determination result to the output device.

The output form of the first determination result indicating the presence of incidence of the disease may be any of character information indicating “presence of disease incidence”, a symbol such as a circle mark, animation, voice, and the like, but is not limited thereto. Similarly, the output form of the first determination result indicating the absence of incidence of the disease may be any of character information indicating “absence of disease incidence”, a symbol such as an X-mark, animation, voice, and the like, but is not limited thereto.

In addition, the output form of the second determination result indicating the presence of disease incidence may be any of character information indicating “presence of disease incidence”, a symbol such as a circle mark, animation, voice, and the like, but is not limited thereto. Similarly, the output form of the second determination result indicating the absence of disease incidence may be any of character information indicating “absence of disease incidence”, a symbol such as an X-mark, animation, voice, and the like, but is not limited thereto.

In addition, in a case where the first determination result based on the first disease incidence probability is “presence of disease incidence” and the second determination result based on the second disease incidence probability is “absence of disease incidence”, the output unit 20E may output, to the output device, output information further including information indicating that there is a possibility of incidence of a type of disease other than the type represented by the second disease incidence probability.

The output unit 20E may output, to the output device, output information further including information indicating a higher risk level with an increasing value of the first disease incidence probability used for determination of the first determination result, together with the first determination result. Similarly, the output unit 20E may output, to the output device, output information further including information indicating a higher risk level with an increasing value of the second disease incidence probability used for determination of the second determination result, together with the second determination result.

The output unit 20E may determine whether the first disease incidence probability estimated by the first disease incidence probability estimation unit 20C belongs to any range obtained by classifying the entire range of the disease incidence probability from the minimum value 0 to the maximum value 1.0 into two or more ranges, and use information indicating the degree of the first disease incidence probability corresponding to the range to which the first disease incidence probability belongs, as the first determination result or the risk level.

Similarly, the output unit 20E may determine whether the second disease incidence probability estimated by the second disease incidence probability estimation unit 20D belongs to any probability range obtained by classifying the entire range of the disease incidence probability from the minimum value 0 to the maximum value 1.0 into two or more ranges, and use information indicating the degree of the second disease incidence probability corresponding to the range to which the second disease incidence probability belongs, as the second determination result or the risk level. For example, the output unit 20E sets in advance a range of the pancreatic cancer incidence probability from 0.9 to 1.0 as risk level A, a range of the pancreatic cancer incidence probability from 0.5 to 0.9 as risk level B, a range of the pancreatic cancer incidence probability from 0.1 to 0.5 as risk level C, and a range of the pancreatic cancer incidence probability from 0.0 to 0.1 as risk level D. Then, for example, when the pancreatic cancer incidence probability “0.2” illustrated in FIG. 6 is estimated, the output unit 20E may output information including risk level C corresponding to the range including 0.2 as the risk level of the second disease incidence probability.

In addition, the output unit 20E may output, to the output device, output information about the first disease incidence probability after correction and the second disease incidence probability after correction obtained by correcting at least one of the first disease incidence probability and the second disease incidence probability based on the first disease incidence probability and the second disease incidence probability.

For example, the first disease incidence probability, which is information indicating the probability of being suffering from the disease estimated by the first disease incidence probability estimation unit 20C, may be less than the second disease incidence probability, which indicates the probability of being suffering from the disease type estimated by the second disease incidence probability estimation unit 20D. In such a case, the output unit 20E corrects at least one of the first disease incidence probability estimated by the first disease incidence probability estimation unit 20C and the second disease incidence probability estimated by the second disease incidence probability estimation unit 20D to satisfy the relationship of first disease incidence probability≥second disease incidence probability.

For example, the output unit 20E corrects the first disease incidence probability estimated by the first disease incidence probability estimation unit 20C to be a value equal to or greater than the second disease incidence probability estimated by the second disease incidence probability estimation unit 20D to satisfy the relationship of first disease incidence probability≥second disease incidence probability.

In addition, the output unit 20E may correct the second disease incidence probability estimated by the second disease incidence probability estimation unit 20D to be a value less than the first disease incidence probability estimated by the first disease incidence probability estimation unit 20C to satisfy the relationship of first disease incidence probability≥second disease incidence probability.

In addition, the output unit 20E may correct both the first disease incidence probability estimated by the first disease incidence probability estimation unit 20C and the second disease incidence probability estimated by the second disease incidence probability estimation unit 20D to satisfy the relationship of first disease incidence probability≥second disease incidence probability.

Then, the output unit 20E may output information about the first disease incidence probability after correction and the second disease incidence probability after correction to the output device.

Next, an example of a procedure of information processing executed by the information processing apparatus 10 of the present embodiment will be described.

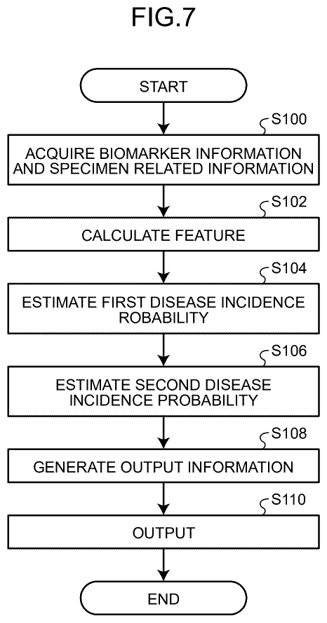

FIG. 7 is a flowchart illustrating the example of the procedure of the information processing executed by the information processing apparatus 10 of the present embodiment.

The processing unit 20 executes processing of steps S100 to S110 each time the biomarker information about one specimen collected from the subject and the specimen related information of the specimen are acquired.

Specifically, the acquisition unit 20A of the processing unit 20 acquires the biomarker information and the specimen related information (step S100). The acquisition unit 20A acquires, for example, the biomarker information and the specimen related information from the external information processing apparatus via the communication unit 12. In addition, the acquisition unit 20A may acquire the biomarker information and the specimen related information stored in the storage unit 16. The acquisition unit 20A may acquire the biomarker information and the specimen related information input according to the operation instruction of the UI unit 14 by the user.

The feature calculation unit 20B calculates the feature of the expression level of the biomarker represented by the biomarker information acquired in step S100 (step S102). For example, when the biomarker information illustrated in FIG. 2A and the specimen related information illustrated in FIG. 2B are acquired in step 100, the feature calculation unit 20B calculates, for example, the feature illustrated in FIG. 2C.

The first disease incidence probability estimation unit 20C estimates the first disease incidence probability of cancer by using the biomarker information acquired in step S100 or the first information that is the feature calculated in step S102 and the specimen related information acquired in step S100 (step S104). Through processing of step S104, for example, the first disease incidence probability estimation unit 20C estimates the first disease incidence probability illustrated in FIG. 4.

The second disease incidence probability estimation unit 20D estimates the second disease incidence probability of a cancer type by using the first disease incidence probability estimated in step S104, the biomarker information acquired in step S100 or the first information that is the feature calculated in step S102, and the specimen related information acquired in step S100 (step S106). Through processing of step S106, for example, the second disease incidence probability estimation unit 20D estimates the second disease incidence probability illustrated in FIG. 6.

Next, the output unit 20E generates output information about the first disease incidence probability estimated in step S104 and the second disease incidence probability estimated in step S106 (step S108). Then, the output unit 20E outputs the output information generated in step S108 to the output device (step S110), and ends this routine.

As described above, the processing unit 20 of the information processing apparatus 10 of the present embodiment estimates the first disease incidence probability of the disease based on the first information about the expression levels of the one or more types of biomarkers of the specimen, and estimates the second disease incidence probability of the disease type based on the first information and the first disease incidence probability.

In this manner, the processing unit 20 of the information processing apparatus 10 of the present embodiment does not directly estimate the disease incidence probability of the disease type from the expression level of the biomarker, but instead estimates the first disease incidence probability of the disease and further estimates the second disease incidence probability of the disease type by using the estimated first disease incidence probability. The processing unit 20 of the information processing apparatus 10 of the present embodiment estimates the disease incidence probability in a gradual manner.

Therefore, the processing unit 20 of the information processing apparatus 10 of the present embodiment can improve the estimation performance of the first disease incidence probability of the disease and the second disease incidence probability of the disease type, as compared with the case of estimating the disease incidence probability through processing in a single stage alone. The processing unit 20 estimates, in the first stage, the disease incidence probability (first disease incidence probability) for the disease, and estimates, in the second stage, the disease incidence probability (second disease incidence probability) for the disease type being a more detailed classification by using the first disease incidence probability estimated in the first stage. Therefore, the processing unit 20 of the information processing apparatus 10 of the present embodiment can subdivide the classification problem through the stepwise estimation, and can improve the estimation performance of the disease.

Therefore, the processing unit 20 of the information processing apparatus 10 of the present embodiment can improve the estimation performance of the disease.

In addition, the first information is the expression level of the biomarker or the feature of the expression level of the biomarker. Therefore, the processing unit 20 of the present embodiment can further improve the estimation performance of the disease by using the expression level of the biomarker or the feature of the expression level of the biomarker as the first information.

In addition, the processing unit 20 estimates the first disease incidence probability based on the first information and the specimen related information about the specimen, and estimates the second disease incidence probability based on the first information, the first disease incidence probability, and the specimen related information. Thus, the processing unit 20 of the present embodiment can further improve the estimation performance of the disease by performing the estimation further using the specimen related information.

In addition, the processing unit 20 outputs the output information about the first disease incidence probability and the second disease incidence probability.

Therefore, the processing unit 20 can easily provide the estimation result to the user. In addition, for example, when the processing unit 20 outputs output information including the second disease incidence probability lower than the first disease incidence probability by a predetermined probability or more, the processing unit 20 can provide information indicating that the incidence probability of a specific type of disease represented by the second disease incidence probability is low, whereas the possibility that another type of disease other than the specific type has occurred is high.

In addition, the processing unit 20 outputs the output information about the first disease incidence probability after correction and the second disease incidence probability after correction obtained by correcting at least one of the first disease incidence probability and the second disease incidence probability, based on the first disease incidence probability and the second disease incidence probability.

Therefore, in addition to the above-described effects, the processing unit 20 can output the output information about the disease incidence probability with further higher accuracy.

Next, an example of a hardware configuration of the information processing apparatus 10 according to the above-described embodiment will be described.

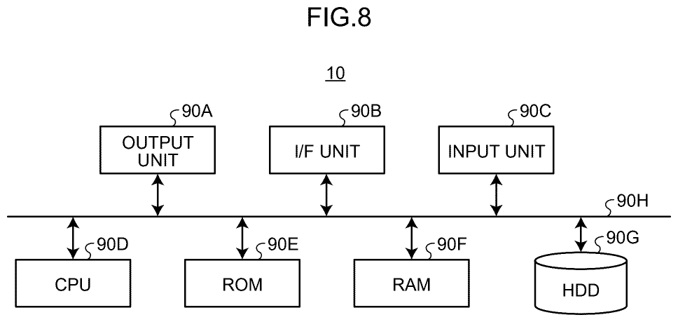

FIG. 8 is a hardware configuration diagram as the example of the information processing apparatus 10 of the above-described embodiment.

The information processing apparatus 10 of the above-described embodiment includes a control device such as a central processing unit (CPU) 90D, a storage device such as a read only memory (ROM) 90E, a random access memory (RAM) 90F, and a hard disk drive (HDD) 90G, an I/F unit 90B that is an interface with various devices, an output unit 90A that outputs various types of information, an input unit 90C that receives an operation by the user, and a bus 90H that connects the units, and has a hardware configuration using a normal computer.

In the information processing apparatus 10 of the above-described embodiment, the CPU 90D reads a computer program from the ROM 90E onto the RAM 90F, and executes the computer program, and as a result, the above-described units are implemented on the computer.

Note that the computer program for executing each of the above-described processing executed by the information processing apparatus 10 of the above-described embodiment may be stored in the HDD 90G. In addition, the computer program for executing each of the above-described processing executed by the information processing apparatus 10 of the above-described embodiment may be provided by being incorporated in the ROM 90E in advance.

The computer program for executing the above-described processing executed by the information processing apparatus 10 of the above-described embodiment may be stored, as a file in an installable format or an executable format, in a computer-readable storage medium such as a CD-ROM, a CD-R, a memory card, a digital versatile disc (DVD), or a flexible disk (FD), and may be provided as a computer program product. In addition, the computer program for executing the above-described processing executed by the information processing apparatus 10 of the above-described embodiment may be stored on a computer connected to a network such as the Internet, and provided by being downloaded via the network. Moreover, the computer program for executing the above-described processing executed by the information processing apparatus 10 of the above-described embodiment may be provided or distributed via the network such as the Internet.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An information processing apparatus comprising
a hardware processor connected to a memory and configured to:
estimate a first disease incidence probability of a disease based on first information about expression levels of one or more types of biomarkers of a specimen, and
estimate a second disease incidence probability of a disease type based on the first information and the first disease incidence probability.

2. The information processing apparatus according to claim 1, wherein the hardware processor is further configured to output, to an output device, output information about the first disease incidence probability and the second disease incidence probability.

3. The information processing apparatus according to claim 2, wherein the hardware processor is further configured to output the output information including at least one of
a first determination result on a presence or absence of incidence of the disease based on the first disease incidence probability, or
a second determination result on the presence or absence of incidence of the disease type based on the second disease incidence probability.

4. The information processing apparatus according to claim 2, wherein the hardware processor is further configured to output the output information including information about the first disease incidence probability after correction and the second disease incidence probability after correction obtained by correcting at least one of the first disease incidence probability and the second disease incidence probability based on the first disease incidence probability and the second disease incidence probability.

5. The information processing apparatus according to claim 1, wherein the first information indicates an expression level of the biomarker or a feature of the expression level of the biomarker.

6. The information processing apparatus according to claim 5, wherein the feature is a relative index of the expression level of the biomarker with respect to a reference index.

7. The information processing apparatus according to claim 1, wherein the biomarker is an miRNA.

8. The information processing apparatus according to claim 1, wherein
the disease is cancer, and
the disease type is a cancer type.

9. The information processing apparatus according to claim 1, wherein the hardware processor is further configured to estimate the second disease incidence probability of each of disease types based on the first information and the first disease incidence probability.

10. The information processing apparatus according to claim 1, wherein the hardware processor is further configured to
perform the estimation of the first disease incidence probability based on the first information and specimen related information about the specimen, and
perform the estimation of the second disease incidence probability based on the first information, the first disease incidence probability, and the specimen related information.

11. The information processing apparatus according to claim 1, wherein the hardware processor is further configured to
perform the estimation of the first disease incidence probability from the first information by using a first learned model, and
perform the estimation of the second disease incidence probability from the first information and the first disease incidence probability by using a second learned model.

12. An information processing method implemented by a computer, the method comprising:
estimating a first disease incidence probability of a disease based on first information about expression levels of one or more types of biomarkers of a specimen; and
estimating a second disease incidence probability of a disease type based on the first information and the first disease incidence probability.

13. A computer program product comprising a non-transitory computer readable recording medium on which a computer program executable by a computer is stored, the computer program instructing the computer to perform processing, the processing including:
estimating a first disease incidence probability of a disease based on first information about expression levels of one or more types of biomarkers of a specimen; and
estimating a second disease incidence probability of a disease type based on the first information and the first disease incidence probability.
